(19) United States
(12) Patent Application Publication
(10) Pub. No.: US 2021/0379090 A1
(43) Pub. Date: Dec. 9, 2021

(54) REMDESIVIR ORAL DELIVERY SYSTEM

(71) Applicant: ABON PHARMACEUTICALS, LLC, Northvale, NJ (US)

(72) Inventors: Salah U. AHMED, New City, NY (US); Yanming ZU, Tenafly, NJ (US); Hetalben PRAJAPATI, Dumont, NJ (US); Mohammed I. SYED, Dumont, NJ (US); Tahseen A. CHOWDHURY, Township Of Washington, NJ (US)

(21) Appl. No.: 17/340,862

(22) Filed: Jun. 7, 2021

Related U.S. Application Data
(60) Provisional application No. 63/036,712, filed on Jun. 9, 2020.

(51) Int. Cl. A61K 31/675; A61K 9/48; A61K 9/00

(52) U.S. Cl. CPC A61K 31/675; A61K 9/4858; A61K 9/4866; A61K 9/4833; A61K 9/4891; A61K 9/0053

(57) ABSTRACT

The present disclosure provides a remdesivir oral delivery system bypassing the first pass hepatic metabolism. More specifically, the disclosure relates to an oral dosage form of remdesivir comprising a lipid based vehicle in an enteric capsule designed to be delivered at intestine to be absorbed by lymphatic pathway, therefore minimizing the first pass hepatic metabolism and improving the oral bioavailability. In some embodiments, the disclosure provides an oral dosage form comprising: (a) remdesivir; (b) a lipid-based vehicle comprising a lipophilic vehicle, an amphiphilic vehicle, a none-aqueous hydrophilic vehicle, or combinations thereof; and (c) an enteric capsule; wherein the remdesivir is dissolved or dispersed in the lipid-based vehicle; and, wherein the remdesivir and the lipid-based vehicle are in the enteric capsule. It also relates to methods of designing and making this dosage form, and methods of usage of this dosage form in the early treatment and prophylaxis of coronavirus infections, e.g., COVID-19.

### FIELD OF THE INVENTION

The present disclosure provides a remdesivir oral delivery system bypassing the first pass hepatic metabolism. More specifically, the disclosure relates to an oral dosage form of remdesivir comprising a liquid based vehicle in an enteric capsule designed to be delivered at intestine to be absorbed by lymphatic pathway, therefore minimizing the first pass hepatic metabolism and improving the oral bioavailability. In some embodiments, the disclosure provides an oral dosage form comprising: (a) remdesivir; (b) a lipid-based vehicle comprising a lipophilic vehicle, an amphiphilic vehicle, a non-aqueous hydrophilic vehicle, or combinations thereof; and (c) an enteric capsule; wherein the remdesivir is dissolved or dispersed in the lipid-based vehicle; and, wherein the remdesivir and the lipid-based vehicle are in the enteric capsule. It also relates to methods of designing and making this dosage form, and methods of usage of this dosage form in the early treatment and prophylaxis of coronavirus infection, e.g., COVID-19.

### BACKGROUND

Coronavirus infection, e.g., COVID-19, is a contagious infectious disease caused by a newly emerged coronavirus (SARS-CoV-2). Both seasonal flu viruses and SARS-CoV-2 are contagious viruses that cause respiratory illness. However, some coronaviruses, e.g., SARS-CoV-2, can be more contagious and have higher mortality rate compared to the flu. Ro is a measure used to determine how easily a virus spreads, which is an estimate of the average number of people who catch the virus from a single infected person. The Ro value for the flu was reported to be about 1.3 while the Ro for SARS-CoV-2 is reported to be about 5.7. In the U.S., the death rate from seasonal flu is usually about 0.1%. By contrast, among reported COVID-19 cases in the U.S., nearly 6% have died.

There are several drugs that can be prescribed to treat flu illness. Antiviral drugs for flu come in the form of pills, liquid, inhaled powder, or intravenous solution. Flu antiviral drugs can make flu symptoms milder and can shorten duration of illness. Antiviral drugs work best if started soon after getting sick (within two days of symptoms starting). They may also prevent serious flu complications. For people with high-risk factors, treatment with an antiviral drug can mean the difference between having a milder illness versus a very serious illness that could result in a hospital stay, or death. CDC recommends antiviral drugs to treat, as early as possible, confirmed or suspected flu in people who are severely ill and people who are at high risk of serious flu complications who develop flu symptoms. See, e.g., Influenza (Flu), https://www.cdc.gov/flu/index.htm. The flu antiviral drugs, such as Tamiflu, are indicated for both the treatment and prophylaxis of the flu viruses.

While several drugs have been developed to treat flu illness, currently there is only one antiviral drug, remdesivir, authorized for emergency use for the treatment of coronavirus (SARS-CoV-2) infection. Unfortunately, this anti-coronavirus drug remdesivir is only available as injection dosage forms for intravenous (IV) infusion, which requires administration and medical supervision by a healthcare provider in an in-patient hospital setting. The remdesivir injections for IV infusion are authorized for use under an Emergency Use Authorization (EUA) by the U.S. Food and Drug Administration (FDA) for treatment of patients hospitalized with suspected or laboratory confirmed SARS-CoV-2 infection and severe disease. Severe disease is defined as patients with an oxygen saturation (SpO2)≤94% on room air or requiring supplemental oxygen or requiring mechanical ventilation or requiring extracorporeal membrane oxygenation (ECMO). Specifically, remdesivir is only authorized for hospitalized adult and pediatric patients for whom use of an intravenous agent is clinically appropriate.

Currently, two remdesivir formulations have been developed, both being intravenous (IV) infusions. Since remdesivir is practically insoluble in water, betadex sulfobutyl ether sodium (β-cyclodextrin) is used in both formulations as a solubilizing agent. One formulation is a sterile, preservative-free lyophilized solid that is reconstituted in sterile water for injection and diluted with saline prior to intravenous (IV) administration. The second formulation is a sterile, preservative-free solution that is diluted with saline prior to intravenous (IV) administration. The first formulation remdesivir should be stored below 30° C. until used, while the second formulation should be stored at refrigerated temperatures (2° C. to 8° C.) until used.

According to European Medicines Agency (EMA)'s summary on compassionate use for remdesivir (Gilead), “remdesivir is not suitable for oral delivery as its poor hepatic stability would likely result in almost complete first-pass clearance.”

### SUMMARY OF THE INVENTION

The present disclosure provides an oral dosage form of remdesivir which comprises a liquid based vehicle in an enteric capsule designed to be delivered at the intestine to be absorbed by lymphatic pathway, therefore minimizing the first pass hepatic metabolism and improving the oral bioavailability.

In some embodiments, the disclosure provides an oral delivery system for lymphatic absorption comprising remdesivir, wherein remdesivir includes the compound of Formula I and its metabolites, the compound of Formula II and the compound of Formula III, as well as their respective salt forms, stereoisomers, prodrugs and esters thereof.

The present disclosure provides an oral dosage form comprising remdesivir, for the convenient, early and effective treatment of COVID-19, not requiring in-patient hospital setting as needed for the injection dosage forms.

In some embodiments, the disclosure provides an oral dosage form comprising: (a) remdesivir; (b) a lipid-based vehicle comprising a lipophilic vehicle, an amphiphilic vehicle, a non-aqueous hydrophilic vehicle, or combinations thereof; and (c) an enteric capsule, wherein the remdesivir is dissolved or dispersed in the lipid-based vehicle; and wherein the remdesivir and the lipid-based vehicle are in the enteric capsule.

In some embodiments, the remdesivir is about 3% to about 30% (w/w) of the oral dosage form.

In some embodiments, the lipophilic vehicle is about 20% to about 90% (w/w) of the oral dosage form.

In some embodiments, the amphiphilic vehicle is about 10% to about 50% (w/w) of the oral dosage form.

In some embodiments, the non-aqueous hydrophilic vehicle is about 10% to about 50% (w/w) of the oral dosage form.

In some embodiments, the lipophilic vehicle comprises a fatty acid, a medium chain glyceride, a vegetable oil, and combinations thereof. In some embodiments, the fatty acid comprises oleic acid, palmitic acid, stearic acid, linoleic acid, omega-3 and omega-6 fatty acids, and combinations thereof. In some embodiments, the medium chain glyceride comprises Capmul, MCM, and Miglyol. In some embodiments, the vegetable oil comprises castor oil, cottonseed oil, coconut oil, flaxseed oil, hemp seed oil, olive oil, peanut oil, palm seed oil, safflower oil, sesame oil, soybean oil, sunflower oil, and combinations thereof. In some embodiments, the lipophilic vehicle comprises of combinations with lipophilic surfactant such as propylene glycol monocaprylate, propylene glycol monolaurate, polyglyceryl-3 dioleate, sorbitan esters, and combinations thereof.

In some embodiments, the amphiphilic vehicle comprises sorbitan esters, polyethoxylated sorbitan esters, unsaturated polyglycolized glycerides, fatty alcohol ethoxylates, fatty acid ethoxylates, castor oil based ethoxylates, PEG-15 hydroxy stearate, saturated polyglycolized glycerides, propylene glycol esters, PEG-8 caprylic/capric glycerides, polyoxyethylene-polyoxypropylene block copolymers, vitamin E TPGS, and combination thereof.

In some embodiments, the non-aqueous hydrophilic vehicle comprises ethanol, polyethylene glycol, propylene glycol, glycerin, N-methyl-2-pyrrolidone, dimethylacetamide, diethylene glycol monoethyl ether and combinations thereof.

In some embodiments, the enteric capsule starts to dissolve above about pH 5. In some embodiments, the enteric capsule shell begins to dissolve above pH approximately 5.5.

In some embodiments, the disclosure provides a method of making an oral dosage form comprising remdesivir, the method comprising: (a) dissolving the remdesivir in a lipid-based vehicle comprising a lipophilic vehicle, an amphiphilic vehicle, a non-aqueous hydrophilic vehicle, or combinations thereof to form a composition; and (b) encapsulating the composition into a capsule.

In some embodiments, the disclosure provides a method of making an oral dosage form comprising remdesivir, the method comprising: (a) dissolving the remdesivir, in a non-aqueous hydrophilic vehicle to form a remdesivir/hydrophilic vehicle composition; (b) dispersing the remdesivir/hydrophilic vehicle composition into a lipophilic vehicle, an amphiphilic vehicle, or combinations thereof to form a second composition; (a) encapsulating the second composition into a capsule.

In some embodiments, the disclosure provides a method of making an oral dosage form comprising remdesivir, the method comprising: (a) dispersing the remdesivir in a lipid-based vehicle comprising a lipophilic vehicle, an amphiphilic vehicle, a non-aqueous hydrophilic vehicle, or combinations thereof to form a composition; (b) processing the remdesivir lipid-based vehicle composition with a high shear homogenizer to form a homogenous fine-dispersed remdesivir lipid-based composition; and (c) encapsulating the homogenous fine-dispersed remdesivir lipid-based composition into a capsule.

In some embodiments, the disclosure provides a method of making an oral dosage form comprising remdesivir, the method comprising: (a) dispersing the remdesivir in a lipid-based vehicle comprising a lipophilic vehicle, an amphiphilic vehicle, or combinations thereof to form a composition; (b) processing the remdesivir lipid-based vehicle composition with a high shear Dispax-Reactor or a Colloid Mill to form a homogenous fine-dispersed remdesivir lipid-based composition; and (c) encapsulating the homogenous fine-dispersed remdesivir lipid-based composition into a capsule.

In some embodiments, the disclosure provides a method of making an oral dosage form comprising remdesivir, the method comprising: (a) dispersing the remdesivir in a lipophilic vehicle to form a remdesivir/lipophilic vehicle composition; (b) processing the remdesivir/lipophilic vehicle composition with a high shear homogenizer to form a homogenous fine-dispersed remdesivir lipid-based composition; and (c) encapsulating the homogenous fine-dispersed remdesivir lipid-based composition into a capsule.

In some embodiments, the capsule comprises an enteric coating. In some embodiments, the method further comprises adding an enteric coating to the capsule.

In some embodiments, the oral dosage is further packaged in blister or in bottle in an amount sufficient for administration for greater than 5 days. In some embodiments, the oral dosage is further packaged in blister or in bottle in an amount sufficient for administration for 5 to 10 days.

In some embodiments, the oral dosage form as described herein is stable at refrigerated condition. In some embodiments, the oral dosage form is stable at room temperature.

In some embodiments, the disclosure provides a method of treating a coronavirus infection in a subject, the method comprising administering the oral dosage form as described herein. In some embodiments, the administration of the oral dosage form begins at the onset of symptoms associated with coronavirus. In some embodiments, the administration continues daily for at least on week. In some embodiments, the administration continues until the symptoms associated with coronavirus are reduced. In some embodiments, the administration continues until the symptoms associated with coronavirus are substantially eliminated.

In some embodiments, the disclosure provides a method for preventing a coronavirus infection in a subject, the method comprising administering the oral dosage form as described herein. In some embodiments, the coronavirus infection is SARS-CoV-2.

### DETAILED DESCRIPTION OF THE INVENTION

The present disclosure provides to an oral dosage form of remdesivir comprising a liquid based vehicle in an enteric capsule designed to be delivered at intestine to be absorbed by lymphatic pathway, therefore minimizing the first pass hepatic metabolism and improving the oral bioavailability.

The present disclosure provides an oral dosage form comprising remdesivir, for the convenient, early and effective treatment of COVID-19, not requiring in-patient hospital setting as needed for the injection dosage forms. The currently available formulations of remdesivir are injection dosage forms, which provide a significant hurdle for the convenient, early and effective treatment of a large population, likely to be infected by COVID-19. The present disclosure provides for an oral dosage form comprising remdesivir, an adenosine nucleoside prodrug. Remdesivir is a broad spectrum antiviral medication developed by Gilead Sciences, Inc. (Foster City, Calif.), as represented by the compound of Formula I:

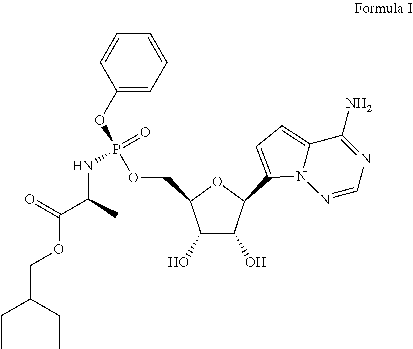 [SMILES: CCC(CC)COC(=O)[C@H](C)N[P@](=O)(OC[C@H]1O[C@@H](c2ccc3c(N)ncnn23)[C@H](O)[C@@H]1O)Oc1ccccc1]

Remdesivir (GS-5734) is a phosphoramidate prodrug of a nucleotide (ProTide) that is able to diffuse into cells where it is converted to nucleoside analog GS-441524 monophosphate via actions of esterases and phophoamidases, which is subsequently phosphorylated to its pharmacologically active metabolite triphosphate GS-443902 by nucleoside-phosphatase kinases. The nucleoside analog GS-441524 is represented by the compound of Formula II. The active metabolite triphosphate GS-443902 (GS-441524 triphosphate) is represented by the compound of Formula III.

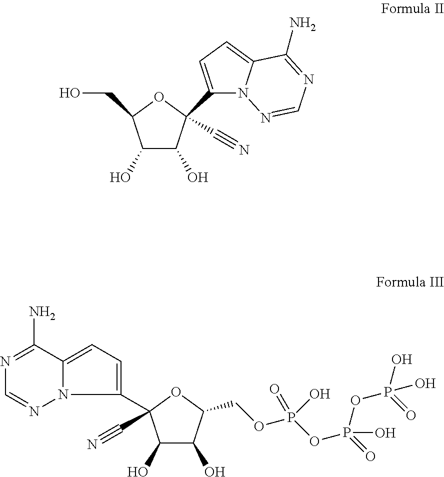 [SMILES: N#C[C@@]1(c2ccc3c(N)ncnn23)O[C@H](CO)[C@@H](O)[C@H]1O ; N#C[C@@]1(c2ccc3c(N)ncnn23)O[C@H](COP(=O)(O)OP(=O)(O)OP(=O)(O)O)[C@@H](O)[C@H]1O]

Thus, in some embodiments, as used herein “remdesivir” includes the compound of Formula I and its metabolites, the compound of Formula II and the compound of Formula III, as well as their respective salt forms, stereoisomers, prodrugs and esters thereof.

In some embodiments, as used herein “remdesivir” includes esters of the compound of Formula II, such as caproate, cypionate, decanoate, enanthate, isobutyrate, isocaproate, phenylpropionate, propionate, undecanoate, palmitate, and succinate etc. ester of the nucleoside analog GS-441524 thereof.

The active metabolite of remdesivir is reported to interfere with the action of viral RNA-dependent polymerase and evades proofreading by viral exoribonuclease (ExoN), causing a decrease in viral RNA production. It is reported that in some viruses, remdesivir causes the RNA-dependent RNA polymerases to pause, but its predominant effect is reported to induce an irreversible chain termination. Unlike with many other chain terminators, this is not mediated by preventing addition of the immediately subsequent nucleotide, but is instead delayed, occurring after five additional bases have been added to the growing RNA chain. It is reported that for the RNA-Dependent RNA Polymerase of MERS-CoV, SARS-CoV-1, and SARS-CoV-2 arrest of RNA synthesis occurs after incorporation of three additional nucleotides. Thus, in some embodiments, the disclosure is directed to a method of arresting RNA synthesis of a virus in a subject, the method comprising administering the dosage form of the present invention to the subject.

A Gilead phase 3 clinical study revealed that hospitalized COVID-19 patients who received remdesivir early within 10 days of symptom onset had improved outcomes compared with those treated late after more than 10 days of symptoms. Therefore, similar to the treatment of flu viruses, remdesivir should work best if started soon after getting sick (within two days of symptoms starting). Similarly, remdesivir may also prevent serious complications caused by the virus. For people with high-risk factors, early treatment with remdesivir, as in an oral dosage form without any requirement for in-patient hospital setting, could mean the difference between having a milder illness versus a very serious illness that could result in a hospital stay, or death. Thus, early treatment of COVID-19 patients with remdesivir oral delivery system offers tremendous benefits, especially in reducing hospitalization or death rate.

It was previously reported that remdesivir is not suitable for oral delivery as its poor hepatic stability would likely result in almost complete first-pass clearance. In order to overcome the challenge of poor hepatic stability of remdesivir after typical oral administration, the present disclosure provides a remdesivir oral delivery system which bypasses the first pass hepatic metabolism. More specifically, the disclosure provides an oral dosage form of remdesivir comprising a liquid based vehicle in an enteric coated soft gel capsule designed to be absorbed by the lymphatic pathway, therefore minimizing the first pass hepatic metabolism and improving the oral bioavailability. The present invention also provides methods of designing and making this dosage form, and methods of using this dosage form in the treatment and prophylaxis of viruses, e.g., RNA viruses, e.g., coronavirus (SARS-CoV-2).

The lymphatic system is comprised of a complex network of channels that carry a clear fluid called lymph. The primary functions of the lymphatic system are reported to maintain the body's water balance by returning extracellular fluid that has leaked out into the interstitial space back to the systemic circulation and to transport immune cells to the lymph nodes. It also reported to play an essential role in absorption of long-chain fatty acids, triglycerides, cholesterol esters, lipid soluble vitamins, and xenobiotics. The primary route for lipid transport is through the intestinal walls via transcellular absorption, paracellular transport, P-glycoprotein, and cytochrome P450 inhibition. Increased production of chylomicrons is also associated with delivery of compounds into the lymphatic system.

The present disclosure provides a method of administering remdesivir via the lymphatic system. Drug delivery via the lymphatic system provides several advantages over the oral absorption via the portal blood. Drugs, e.g., remdesivir, that are transported via the intestinal lymphatic system enter the systemic circulation without first passing through the liver, therefore for those drugs that are highly metabolized on first pass through the liver, the drug delivery via the lymphatic system can increase drug oral bioavailability. Intestinal lymphatic transport has also been reported to be more effective for immunomodulatory and chemotherapeutic drugs since the lymphatic system is the primary route for metastasis of solid tumors and the transport pathway for T and B lymphocytes. Moreover, it has been suggested recently that the development, sequestration, and spread of the human immunodeficiency virus (HIV), hepatitis B and C virus, morbillivirus, canine distemper virus, and severe acute respiratory syndrome (SARS) associated coronavirus, are in association with lymphocytes present in the lymph and lymphoid tissue. See, e.g., J. A. Yañez, et al., Adv Drug Deliv Rev. 2011 Sep. 10; 63(10): 923-942.

Thus, the present disclosure provides a remdesivir oral dosage form which delivers remdesivir via the lymphatic absorption pathway, thereby offering a number of advantages. First, the remdesivir oral dosage form delivers remdesivir via the lymphatic absorption pathway, thereby bypassing the liver, minimizing the first-pass metabolism of remdesivir in the liver, increasing oral remdesivir bioavailability, and therefore making delivery of remdesivir via the oral route feasible. Secondly, the delivery of remdesivir via the lymphatic system can target drug delivery to the coronavirus (COVID-19) in the lymphocytes present in the lymph and lymphoid tissue, and therefore increase the drug antiviral efficacy. Furthermore, the fact of lymphatic system being the transport pathway for T and B lymphocytes (the major immune response cells) may also increase remdesivir efficacy in terms of controlling immune response to the coronavirus, such as COVID-19. It is reported that temporarily suppressing the body's immune system during the early stages of COVID-19 could help a patient avoid severe symptoms. See, e.g., Timing of immune response to COVID-19 may contribute to disease severity https://www.sciencedaily.com/releases/2020/05/200501184320.htm). Thus, in some embodiments, the present disclosure provides a method of administering remdesivir to a subject via the lymphatic system. In some embodiments, the disclosure provides a dosage form suitable for delivering remdesivir via the lymphatic system.

Various lipid-based drug delivery systems (LBDDS) were previously reported for the lymphatic drug delivery via the oral routes, such as oil solutions, emulsions, dispersions, micelles, self-emulsifying drug delivery systems (SEDDS), and self-microemulsifying drug delivery systems (SMEDDS). See, e.g., Chaudhary S, et al., J Drug Target. 2014 December; 22(10):871-82. However, it is well recognized there is a prerequisite for the drug to be used effectively in the lipid-based drug delivery systems, i.e., lipophilicity of the drug, typically reflected by the Log D or Log P value of the drug. Based on Browne et al.'s review and analysis of FDA approved drugs using lipid-based formulations, all the approved drugs in various lipid-based drug delivery systems are lipophilic compounds, with a median Log P of 4.85 and an interquartile Log P range of 3.66-5.97. See, e.g., Savla R, et al., Drug Dev Ind Pharm. 2017 November; 43(11):1743-1758.

Remdesivir has a relatively poor lipophilicity with a low Log D value of 2.1, and therefore based on lipophilicity appears to fail to meet the high lipophilicity prerequisite used in the lipid-based drug delivery systems, based on the analysis of FDA approved drugs using lipid-based formulations. The poor lipophilicity of remdesivir made the lymphatic system delivery for remdesivir in the lipid-based delivery system challenging.

To compound the difficulties, the storage condition of the previously reported remdesivir solution indicates stability issues of remdesivir in its solution state, requiring storage at refrigerated temperatures (2° C. to 8° C.) and following dilution with 0.9% saline, storage for only up to 4 hours at room temperature (20° C. to 25° C.) or 24 hours at refrigerated temperatures (2° C. to 8° C.). The storage condition of remdesivir solution indicates stability issues of remdesivir in its solution state. The apparent instability of remdesivir provides another challenge to develop a stable lymphatic oral dosage form.

One additional obstacle to lymphatic delivery is that chylomicrons absorption occurs in the absorptive enterocyte of the small intestine. Thus, the oral dosage forms comprising remdesivir as described herein provide a method of delivery the remdesivir to the small intestine, thereby avoiding premature breakdown of the dosage form before reaching its target absorption site in the gastrointestinal tract, maximizing its availability and/or absorption at the target absorption site via the lymphatic system absorption, and therefore further increasing its oral bioavailability.

In some embodiments, the disclosure provides an oral delivery system. The term “oral delivery system” refers to any means for delivering the remdesivir to the gastrointestinal tract. The delivery system can be of any shape or size suitable for administration. In some embodiments, the oral delivery system can be a capsule. In some embodiments, the oral delivery system can be a hard capsule. In some embodiments, the oral delivery system can be a soft capsule. In some embodiments, the oral delivery system can be a soft gel capsule. In some embodiments, the oral delivery system can be an enteric coated capsule.

In some embodiments, the oral delivery system of remdesivir comprises the drug or its derivatives with pharmaceutically acceptable excipients.

As used herein, “derivatives” of remdesivir includes the compound of Formula I and its metabolites, the compound of Formula II and the compound of Formula III, as well as their respective salt forms, stereoisomers, prodrugs and esters thereof.

As used herein, “pharmaceutically acceptable excipients” refers to any component of the oral delivery system that aids in manufacture of remdesivir, influences solubility, pharmacokinetics, or physiological absorption and is either approved for use in human subjects or generally recognized as safe by regulatory agencies such as the U.S. Federal Drug Administration.

In some embodiments, the disclosure provides an oral delivery system for lymphatic absorption comprising remdesivir, wherein remdesivir includes the compound of Formula I and its metabolites, the compound of Formulate II and the compound of Formula III, as well as their respective salt forms, stereoisomers, prodrugs and esters thereof.

In some embodiments, the disclosure provides an oral dosage form. The term “oral dosage form” refers to any means for delivering the remdesivir to the gastrointestinal tract. The dosage form can be of any shape or size suitable for administration. In some embodiments, the oral dosage form can be a capsule. In some embodiments, the oral dosage form can be a hard capsule. In some embodiments, the oral dosage from can be a soft capsule. In some embodiments, the oral dosage from can be a soft gel capsule. In some embodiments, the oral dosage form can be an enteric coated capsule.

The dosage forms of the present disclosure comprise a lipid-based vehicle. Lipid-based vehicles can include any lipid suitable for dissolving or suspending the remdesivir. Lipids are commonly known as esters of fatty acids—lipophilic hydrocarbon chains linked to a hydrophilic group like glycerol, polyglycerol, or polyalcohol. The melting range, solubilization capacity, and miscibility properties of the excipient are defined by the fatty acid chain length and degree of unsaturation. The amphiphilicity or dual polar and non-polar nature of lipids is characterized by the Hydrophilic Lipophilic Balance (HLB), a measure of the excipient dispersibility in aqueous media. In some embodiments, the remdesivir is dissolved, i.e., substantially in solution, in the lipid-based vehicle. In some embodiments, the remdesivir is dispersed, e.g., in a homogeneous suspension, in the lipid-based vehicle. In some embodiments, the dissolved or dispersed remdesivir/lipid-based vehicle is transported via lipid transport through the intestinal walls via transcellular absorption, forming chylomicrons for delivery of the active compounds into the lymphatic system.

In some embodiments, the lipid-based vehicle comprises a lipophilic vehicle. In some embodiment, the lipophilic vehicle is pharmaceutically acceptable. In some embodiments, the lipophilic vehicle comprises, mono- and di-glycerides, and oils. In some embodiments, the lipophilic vehicle comprises a fatty acid, a medium chain glyceride, e.g., a medium chain glyceride, a vegetable oil, and combinations thereof. In some embodiments, the fatty acid is saturated. In some embodiments, the fatty acid is unsaturated, e.g., mono-unsaturated, di-unsaturated or polyunsaturated. In some embodiments, the fatty acid comprises oleic acid, palmitic acid, stearic acid, linoleic acid, omega-3 and omega-6 fatty acids, and combinations thereof. In some embodiments, the fatty acid is a C6-C24 saturated fatty acid. In some embodiments, the fatty acid is a C6-C15 fatty acid, e.g., C6-C12 fatty acid, e.g., a C6 (hexanoic acid), C7 (heptanoic acid), C8 (octanoic acid, C9 (nonanoic acid), C10 (decanoic acid), C11 (undecanoic acid), or C12 (dodecanoic acid) fatty acid. In some embodiments, the fatty acid is an unsaturated fatty acid. In some embodiments, the fatty acid is a C6-C24 unsaturated fatty acid. In some embodiments, the unsaturated fatty acid is a C6-C15 unsaturated fatty acid, e.g., C6-C12 unsaturated fatty acid. In some embodiments, the unsaturated fatty acid is myristoleic acid, palmitoleic acid, sapienic acid, oleic acid, elaidic acid, vaccenic acid, linoleic acid, linoelaidic acid, α-linolenic acid, arachidonic acid, eicosapentaenoic acid, erucic acid, docosahexaenoic acid or combinations thereof.

In some embodiments, the lipophilic vehicle comprises a diglyceride or triglyceride. In some embodiments, the diglyceride or triglyceride is saturated. In some embodiments, the diglyceride or triglyceride is unsaturated. In some embodiments, the diglyceride comprises one, or two medium chain fatty acids, i.e., C6 to C12 fatty acids. In some embodiments, the triglyceride comprises one, two, or three medium chain fatty acids, i.e., C6 to C12 fatty acids. In some embodiments, the medium chain triglycerides comprise a mixture of triglycerides of varying chain length, e.g., in some embodiments, the medium chain glyceride comprises Capmul®, MCM and Miglyol®. In some embodiments, the medium chain triglyceride comprises a vegetable oil. In some embodiments, the vegetable oil comprises castor oil cottonseed oil, olive oil, peanut oil, safflower oil, sesame oil, soybean oil, sunflower oil, coconut oil, palm seed oil and combinations thereof. In some embodiments, the lipophilic vehicle comprises of combinations with lipophilic surfactant such as propylene glycol monocaprylate, propylene glycol monolaurate, polyglyceryl-3 dioleate, sorbitan esters, and combinations thereof.

In some embodiments, the lipid-based vehicle comprises an amphiphilic vehicle. In some embodiments, the amphiphilic vehicle comprises sorbitan esters, polyethoxylated sorbitan esters, unsaturated polyglycolized glycerides, fatty alcohol ethoxylates, fatty acid ethoxylates, castor oil based ethoxylates, PEG-15 hydroxy stearate, saturated polyglycolized glycerides, propylene glycol esters, PEG-8 caprylic/capric glycerides, polyoxyethylene-polyoxypropylene block copolymers, vitamin E TPGS, and combinations thereof.

In some embodiments, the lipid-based vehicle comprises a non-aqueous hydrophilic vehicle. In some embodiments, the non-aqueous hydrophilic vehicle comprises ethanol, polyethylene glycol, propylene glycol, glycerin, N-methyl-2-pyrrolidone, dimethylacetamide, diethylene glycol monoethyl ether and combinations thereof.

In some embodiments, the lipid-based vehicle comprises a combination of a lipophilic vehicle, an amphiphilic vehicle, and a non-aqueous hydrophilic vehicle. In some embodiments, the lipid-based vehicle comprises a combination of a lipophilic vehicle, and an amphiphilic vehicle. In some embodiments, the lipid-based vehicle comprises a combination of a lipophilic vehicle and a non-aqueous hydrophilic vehicle. In some embodiments, the lipid-based vehicle comprises a combination of an amphiphilic vehicle and a non-aqueous hydrophilic vehicle. In some embodiments, the lipid-based vehicle comprises only a lipophilic vehicle, an amphiphilic vehicle, or a non-aqueous hydrophilic vehicle.

In some embodiments, the dosage form of the present invention does not comprise β-cyclodextrin.

In some embodiments, the oral dosage form can comprise various concentrations of remdesivir. In some embodiments, the remdesivir is about 3% to about 30% (w/w) of the oral dosage form. In some embodiments, the remdesivir is about 5% to about 25%, about 6% to about 18% (w/w), about 8% to about 16% (w/w), about 10% to about 14% (w/w) or about 12% (w/w) of the oral dosage form.

In some embodiments, the oral dosage form can comprise various concentrations of the lipid-based vehicle. In some embodiments, the lipid-based vehicle is about 70% to about 97% (w/w) of the oral dosage form. In some embodiments, the lipid-based vehicle is about 80% to about 97% (w/w), or about 90% to about 97% (w/w), of the oral dosage form.

In some embodiments, the oral dosage form can comprise various concentrations of the lipophilic vehicle. In some embodiments, the lipophilic vehicle is about 20% to about 90% (w/w) of the oral dosage form. In some embodiments, the lipophilic vehicle is about 30% to about 90% (w/w), about 40% to about 90% (w/w), about 50% to about 90% (w/w), about 60% to about 90% (w/w), or about 70% to about 90% (w/w) of the oral dosage form.

In some embodiments, the oral dosage form can comprise various concentrations of the amphiphilic vehicle. In some embodiments, the amphiphilic vehicle is about 10% to about 50% (w/w) of the oral dosage form. In some embodiments, the amphiphilic vehicle is about 20% to about 50% (w/w), about 20% to about 40% (w/w), or about 20% to about 30% (w/w) of the oral dosage form.

In some embodiments, the oral dosage form can comprise various concentrations of the non-aqueous hydrophilic vehicle. In some embodiments, the non-aqueous hydrophilic vehicle is about 10% to about 50% (w/w) of the oral dosage form. In some embodiments, the non-aqueous hydrophilic vehicle is about 10% to about 40% (w/w) of the oral dosage form. In some embodiments, the non-aqueous hydrophilic vehicle is about 10% to about 30% (w/w), about 20% to about 30% (w/w), or about 10% to about 25% (w/w) of the oral dosage form.

In some embodiments, the ratio of non-aqueous hydrophilic vehicle to lipophilic vehicle is about 1:8 to about 5:1, about 1:5 to about 2.5:1 or about 1:2 to about 2:1. In some embodiments, the ratio of non-aqueous hydrophilic vehicle to lipophilic vehicle is about 1.5:1 to 1:1.5 or about 1:1.

In some embodiments, the ratio of non-aqueous hydrophilic vehicle to amphiphilic vehicle is about 1:4 to about 5:1, about 1:2 to about 3:1 or about 0.5:1 to 2:0.5. In some embodiments, the ratio of non-aqueous hydrophilic vehicle to amphiphilic vehicle is about 4.5:1 to 6:1 or about 5:1.

In some embodiments, the ratio of lipophilic vehicle to amphiphilic vehicle is about 1:4 to about 5:1, about 1:2 to about 3:1 or about 0.5:1 to 2:0.5. In some embodiments, the ratio of lipophilic vehicle to amphiphilic vehicle is about 4.5:1 to 6:1 or about 5:1.

In some embodiments, the dosage form comprises one of the concentrations in Table A:

TABLE A

| | Concentrations (w/w) | | | | | | |
|---|---|---|---|---|---|---|---|
| | 1 | 2 | 3 | 4 | 5 | 6 | 7 |
| Non-aqueous | 10%- | 15%- | 20%- | 25%- | 10%- | 20%- | 20%- |
| hydrophilic | 50% | 45% | 45% | 35% | 40% | 40% | 45% |
| vehicle | | | | | | | |
| Lipophilic | 200%- | 20%- | 20%- | 25%- | 30%- | 30%- | 20%- |
| vehicle | 900% | 80% | 70% | 35% | 80% | 60% | 45% |
| Amphiphilic | 10%- | 15%- | 20%- | 25%- | 10%- | 20%- | 20%- |
| vehicle | 50% | 45% | 45% | 35% | 40% | 40% | 45% |

The oral dosage forms as disclosed herein have an enteric capsule. The term capsule can include, e.g., a soft capsule, a seamless capsule, or a hard capsule. In some embodiments, the capsule is a soft capsule. The term “enteric capsule” refers to a dosage form which does not substantially dissolve in the stomach, but dissolves in the intestines, thereby releasing the contents of the dosage form in the intestine. In some embodiments, the enteric capsule dissolves in the small intestine. In some embodiments, the enteric capsule dissolves in the large intestine. In some embodiments, the capsules of the present disclosure comprise an enteric coating, thereby providing that the capsule does not substantially dissolve in the stomach, but dissolves in the intestines. In some embodiments, the capsule comprises a material which is enteric, i.e., the capsule material does not substantially dissolve in the stomach, but dissolves in the intestines.

In some embodiments, the enteric capsule does not dissolve in a pH below 6. In some embodiments, the enteric capsule does not dissolve in a pH below 5.8, 5.7, 5.6, 5.5, 5.4, 5.3, 5.2 5.1, or 5.0. In some embodiments, the enteric capsule starts to dissolve above about pH 5. In some embodiments, the enteric capsule begins to dissolve above pH approximately 5.5.

Methods of making an enteric capsule are known to the artisan. In some embodiments, the disclosure provides a method of making an oral dosage form comprising remdesivir, the method comprising: (a) dissolving the remdesivir in a lipid-based vehicle comprising a lipophilic vehicle, an amphiphilic vehicle, a non-aqueous hydrophilic vehicle, or combinations thereof to form a composition; and (b) encapsulating the composition into a capsule. Methods of encapsulating are known to the skilled artisan. In some embodiments, the capsule comprises an enteric coating. In some embodiments, the method further comprises adding an enteric coating to the capsule.

In some embodiments, the disclosure provides a method of making an oral dosage form comprising remdesivir, the method comprising: (a) dissolving the remdesivir, in a non-aqueous hydrophilic vehicle to form a remdesivir/hydrophilic vehicle composition; (b) dispersing the remdesivir/hydrophilic vehicle composition into a lipophilic vehicle to form a second composition; (c) encapsulating the second composition into a capsule. In some embodiments, the method comprises dispersing the remdesivir/hydrophilic vehicle composition into a lipophilic vehicle and an amphiphilic vehicle to form a second composition. In some embodiments, the capsule comprises an enteric coating. In some embodiments, the method further comprises adding an enteric coating to the capsule.

In some embodiments, the disclosure provides a method of making an oral dosage form comprising remdesivir, the method comprising: (a) dispersing the remdesivir in a lipid-based vehicle to form a remdesivir/lipid-based vehicle composition; (b) processing the remdesivir/lipid-based vehicle composition with a high shear homogenizer to form a homogenous fine-dispersed remdesivir/lipid-based composition; and (c) encapsulating the homogenous fine-dispersed remdesivir/lipid-based composition into a capsule. In some embodiments, the capsule comprises an enteric coating. In some embodiments, the method further comprises adding an enteric coating to the capsule.

Types of high shear homogenizers are generally known in the art, e.g. a Dispax-Reactor or a Colloid Mill.

In some embodiments, the oral dosage is further packaged in blister or in bottle in an amount sufficient for administration for greater than 5 days. In some embodiments, the oral dosage is further packaged in blister or in bottle in an amount sufficient for administration for 5 to 10 days.

In some embodiments, the oral dosage form as described herein is stable at refrigerated condition. In some embodiments, the oral dosage form is stable at room temperature.

In some embodiments, the disclosure provides a method of treating a viral infection in a subject, the method comprising administering the oral dosage form as described herein. In some embodiments, the viral infection is caused by Ebola virus, Marburg virus, SARS coronavirus, MERS coronavirus, or SARS-CoV-2.

Previous studies reported COVID-19 patients who received remdesivir early within 10 days of symptom onset had improved outcomes compared with those treated late after more than 10 days of symptoms. In some embodiments, the remdesivir may also prevent serious virus caused complications. For people with high-risk factors, early treatment with remdesivir can mean the difference between having a milder illness versus a very serious illness that could result in a hospital stay, or death. Thus, early treatment of COVID-19 patients with remdesivir offers tremendous benefits, especially in reducing hospitalization or death rate.

Therefore, in some embodiments, the disclosure provides for starting administration of the dosage form soon after exhibiting the first symptoms of coronavirus, e.g., COVID-19. In some embodiments, the dosage form is administered within one day, two days, three days, four days, five days, six days, seven days, eight days, nine days or ten days after exhibiting the first symptoms of the corona virus. In some embodiments, the oral dosage form comprising remdesivir is administered before the onset of any symptoms. For example, in some embodiments, the oral dosage form comprising remdesivir can be administered to a subject who may have been exposed to a coronavirus, e.g., SARS-CoV-2, or works in an environment in which there is a high likelihood of being infected with a coronavirus, e.g., SARS-CoV-2, such as a hospital or a facility in which coronavirus, e.g., SARS-CoV-2, infections are known. In some embodiments, the oral dosage form comprising remdesivir can be administered to an immunocompromised subject.

In some embodiments, the disclosure provides a method of treating a coronavirus infection in a subject, the method comprising administering the oral dosage form as described herein. In some embodiments, the administration of the oral dosage form begins at the onset of symptoms associated with coronavirus. In some embodiments, the administration continues daily for at least on week. In some embodiments, the administration continues until the symptoms associated with coronavirus are reduced. In some embodiments, the administration continues until the symptoms associated with coronavirus are substantially eliminated.

In some embodiments, 25 mg-400 mg of remdesivir are administered per day. In some embodiments, 50 mg-300 mg, 75 mg-200 mg or 100 mg-200 mg of remdesivir are administered per day. In some embodiments, 100 mg, 150 mg or 200 mg or remdesivir are administered per day. In some embodiments, the oral dosage form is administered once a day. In some embodiments, the oral dosage form is administered twice a day. In some embodiments, the oral dosage form is administered three times a day.

### EXAMPLES

### Example 1

Representative remdesivir lipid based oral delivery systems according to the present invention are shown in Table 1.

TABLE 1

| Composition | 1 | 2 | 3 | 4 | 5 |
|---|---|---|---|---|---|
| Remdesivir | 50 mg | 50 mg | 50 mg | 50 mg | 100 mg |
| PEG 400 | 330 mg | 220 mg | 210 mg | 180 mg | 530 mg |
| Propylene | 340 mg | 220 mg | 210 mg | 180 mg | — |
| Glycol | | | | | |
| Ethanol | — | 450 mg | 300 mg | 400 mg | 340 mg |
| MCT and/or | 330 mg | 110 mg | 140 mg | 120 mg | 130 mg |
| Oleic Acid | | | | | |
| Vitamin | — | — | 140 mg | — | — |
| E TPGS | | | | | |
| Kolliphore | — | — | — | 120 mg | — |
| RH 40 | | | | | |
| Total | 1,050 mg | 1,050 mg | 1,050 mg | 1,050 mg | 1,100 mg |

### Example 2

Representative remdesivir lipid based oral delivery systems according to the present invention are shown in Table 2.

TABLE 2

| Composition | 6 | 7 | 8 | 9 | 10 |
|---|---|---|---|---|---|
| Remdesivir | 50 mg | 50 mg | 50 mg | 50 mg | 100 mg |
| PEG 400 and/ | 300 mg | 600 mg | 500 mg | 500 mg | 500 mg |
| or Propylene | | | | | |
| Glycol | | | | | |
| Ethanol | — | — | 100 mg | 100 mg | |
| MCT or | 350 mg | 200 mg | 250 mg | 200 mg | 200 mg |
| Oleic Acid | | | | | |
| Kolliphore | 350 mg | 200 mg | 250 mg | 200 mg | 200 mg |
| RH 40 and/ | | | | | |
| or Vitamin | | | | | |
| E TPGS | | | | | |
| Total | 1,050 mg | 1,050 mg | 1,050 mg | 1,050 mg | 1,100 mg |

### Example 3

Representative remdesivir lipid based oral delivery systems according to the present invention are shown in Table 3.

TABLE 3

| Composition | 11 | 12 | 13 | 14 | 15 |
|---|---|---|---|---|---|
| Remdesivir | 100 mg | 100 mg | 100 mg | 100 mg | 200 mg |
| PEG 400 and/ | 100 mg | 200 mg | 300 mg | 100 mg | — |
| or Propylene | | | | | |
| Glycol | | | | | |
| MCT or | 500 mg | 400 mg | 350 mg | 600 mg | 700 mg |
| Oleic Acid | | | | | |
| Kolliphore | 400 mg | 400 mg | 350 mg | 300 mg | 300 mg |
| RH 40 and/ | | | | | |
| or Vitamin | | | | | |
| E TPGS | | | | | |
| Total | 1,100 mg | 1,100 mg | 1,100 mg | 1,100 mg | 1,200 mg |

### Example 4

Representative remdesivir lipid based oral delivery systems according to the present invention are shown in Table 4.

TABLE 4

| Composition | 16 | 17 | 18 | 19 | 20 |
|---|---|---|---|---|---|
| Remdesivir | 50 mg | 50 mg | 50 mg | 50 mg | 100 mg |
| PEG 400 and/ | 500 mg | 300 mg | 200 mg | 300 mg | 400 mg |
| or Propylene | | | | | |
| Glycol | | | | | |
| Ethanol | 100 mg | 200 mg | 200 mg | 300 mg | 300 mg |
| MCT or | 800 mg | 800 mg | 900 mg | 800 mg | 700 mg |
| Oleic Acid | | | | | |
| Kolliphore | 300 mg | 400 mg | 400 mg | 300 mg | 300 mg |
| RH 40 and/ | | | | | |
| or Vitamin | | | | | |
| E TPGS | | | | | |
| Total | 1,750 mg | 1,750 mg | 1,750 mg | 1,750 mg | 1,800 mg |

### Example 5

A representative remdesivir lipid based oral delivery system according to the present invention is shown in Table 5.

TABLE 5

| Composition | 21 | 22 | 23 | 24 | 25 | 26 |
|---|---|---|---|---|---|---|
| Remdesivir | 50 mg | 50 mg | 50 mg | 50 mg | 50 mg | 50 mg |
| Oleic Acid | 800 mg | 100 mg | 100 mg | 800 mg | 100 mg | 100 mg |
| Propylene | 100 mg | 800 mg | 100 mg | 100 mg | 800 mg | 100 mg |
| Glycol | | | | | | |
| Cremophore | 100 mg | 100 mg | 800 mg | — | | |
| RH | | | | | | |
| Vitamin E | — | | | 100 mg | 100 mg | 800 mg |
| TPGS | | | | | | |
| Total | 1050 mg | 1050 mg | 1050 mg | 1050 mg | 1050 mg | 1050 mg |

### Example 6

A representative soft gel capsule shell composition for the remdesivir lipid based oral delivery system according to the present invention is shown in Table 6.

TABLE 6

| Ingredients | Percent (%) |
|---|---|
| Gelatin Type A (200 Bloom) | 40 |
| Methacrylic Acid Copolymer | 35 |
| (Eudragit L 100 55) | |
| Sodium Hydroxide 1N | Q.S to Dissolve Eudragit |
| Glycerin | 9.64 |
| Propylene Glycol | 15 |
| Methyl Paraben | 0.2 |
| Propyl Paraben | 0.1 |
| Ethylene Diamine Tetra Acetic Acid | 0.05 |
| FD&C Red Color | 0.01 |
| Purified Water | Q.S to Dissolve |
| Total | 100 |

### Example 7

A representative soft gel capsule shell composition for the remdesivir lipid based oral delivery system according to the present invention is shown in Table 7.

TABLE 7

| Ingredients | % |
|---|---|
| Gelatin Type A (200 Bloom) | 51.4 |
| HydroxyPropyl Methyl Cellulose | 9.7 |
| Pthalate (HPMCP 55) | |
| Ammonia Solution | Q.S to Dissolve HPMCP 55 |
| Glycerin | 19.24 |
| Propylene Glycol | 19.3 |
| Methyl Paraben | 0.2 |
| Propyl Paraben | 0.1 |
| Ethylene Diamine Tetra Acetic Acid | 0.05 |
| FD&C Red Color | 0.01 |
| Purified Water | Q.S to Dissolve |
| Total | 100 |

### Example 8

A representative soft gel capsule shell composition for the remdesivir lipid based oral delivery system according to the present invention is shown in Table 8.

TABLE 8

| Ingredients | % |
|---|---|
| Gelatin Type A (200 Bloom) | 52.4 |
| HydroxyPropyl Methyl Cellulose | 7.9 |
| acetate Succinate (HPMCAS HF) | |
| Ammonia Solution | Q.S to Dissolve HPMCAS |
| Glycerin | 19.64 |
| Propylene Glycol | 19.7 |
| Methyl Paraben | 0.2 |
| Propyl Paraben | 0.1 |
| Ethylene Diamine Tetra Acetic Acid | 0.05 |
| FD&C Red Color | 0.01 |
| Purified Water | Q.S to Dissolve |
| Total | 100 |

### Example 9

A representative soft gel capsule shell composition for the remdesivir lipid based oral delivery system according to the present invention is shown in Table 9.

TABLE 9

| Ingredients | % |
|---|---|
| Regular Soft Gel Capsule Shell | |
| Gelatin Type A (200 Bloom) | 45 |
| Sorbitol Powder | 15 |
| Glycerin | 3 |
| Propylene Glycol 400 | 2 |
| Titanium Dioxide | 0.2 |
| Methyl Paraben | 0.2 |
| Propyl Paraben | 0.1 |
| Ethylene Diamine Tetracetic Acid (EDTA) | 0.05 |
| F D&C Red Color | 0.01 |
| Purified Water | Q.S. to 100% |
| Total | 100 |
| Enteric Coating | |
| Poly(meth)acrylate (Eudragit L 30 D-55) | 70 |
| Triacetin | 7 |
| Talc | 23 |
| Total | 100 |

1. An oral dosage form of remdesivir comprising:
(a) a lipid-based vehicle comprising a lipophilic vehicle, an amphiphilic vehicle, a non-aqueous hydrophilic vehicle, or combinations thereof; and
(b) an enteric capsule;
wherein the remdesivir is dissolved or dispersed in the lipid-based vehicle;
and, wherein the remdesivir and the lipid-based vehicle are in the enteric capsule.

2. The oral dosage form of claim 1, wherein the remdesivir is about 3% to about 30% (w/w) of the oral dosage form.

3. The oral dosage form of claim 1, wherein the lipophilic vehicle is about 20% to about 90% (w/w) of the oral dosage form.

4. The oral dosage form of claim 1, wherein the amphiphilic vehicle is about 10% to about 50% (w/w) of the oral dosage form.

5. The oral dosage form of claim 1, wherein the non-aqueous hydrophilic vehicle is about 10% to about 50% (w/w) of the oral dosage form.

6. The oral dosage form of claim 1, wherein the lipophilic vehicle comprises a fatty acid, a medium chain glyceride, a vegetable oil, and combinations thereof.

7. The oral dosage form of claim 6, wherein the fatty acid comprises oleic acid, palmitic acid, stearic acid, linoleic acid, omega-3 and omega-6 fatty acids, and combinations thereof.

8. The oral dosage form of claim 6, wherein the medium chain glyceride comprises Capmul, MCM and Miglyol.

9. (canceled)

10. The oral dosage form of claim 6, wherein the lipophilic vehicle comprises of combinations with lipophilic surfactant such as propylene glycol monocaprylate, propylene glycol monolaurate, polyglyceryl-3 dioleate, sorbitan esters, and combinations thereof.

11. The oral dosage form of claim 1, wherein the amphiphilic vehicle selected from the group consisting of sorbitan esters, polyethoxylated sorbitan esters, unsaturated polyglycolized glycerides, fatty alcohol ethoxylates, fatty acid ethoxylates, castor oil based ethoxylates, PEG-15 hydroxy stearate, saturated polyglycolized glycerides, propylene glycol esters, PEG-8 caprylic/capric glycerides, polyoxyethylene-polyoxypropylene block copolymers, vitamin E TPGS, and combinations thereof.

12. The oral dosage form of claim 1, wherein the non-aqueous hydrophilic vehicle comprises ethanol, polyethylene glycol, propylene glycol, glycerin, N-methyl-2-pyrrolidone, dimethylacetamide, diethylene glycol monoethyl ether and combinations thereof.

13. The oral dosage form of claim 1, wherein the enteric capsule starts to dissolve above about pH 5.

14. (canceled)

15. (canceled)

16. (canceled)

17. (canceled)

18. (canceled)

19. (canceled)

19. (canceled)

20. (canceled)

21. A method of making an oral dosage form comprising remdesivir, the method comprising:
(a) dispersing the remdesivir in a lipid-based vehicle comprising a lipophilic vehicle, an amphiphilic vehicle, a non-aqueous hydrophilic vehicle, or combinations thereof to form a composition;
(b) processing the remdesivir/lipid-based vehicle composition with a high shear mixer to form a homogenous remdesivir/lipid-based composition; and
(c) encapsulating the homogenous remdesivir/lipid-based composition into a capsule.

22. The method of claim 21, wherein the capsule comprises an enteric coating.

23. The method of claim 21, further comprising adding an enteric coating to the capsule.

24. The method of claim 21, wherein the oral dosage is further packaged in blister or in bottle in an amount sufficient for administration for greater than 5 days.

25. (canceled)

26. (canceled)

27. (canceled)

28. A method of treating a viral infection in a subject, the method comprising administering the oral dosage form of claim 1.

29. The method of claim 28, wherein the viral infection is caused by Ebola virus, Marburg virus, SARS coronavirus, MERS coronavirus, or SARS-CoV-2.

30. A method of treating a coronavirus infection in a subject, the method comprising administering the oral dosage form of claim 1.

31. (canceled)

32. The method of claim 30, wherein the administration continues daily for at least one week.

33. (canceled)

34. (canceled)

35. (canceled)

36. The method of claim 30, wherein the coronavirus infection is SARS-CoV-2.
